(19) United States
(12) Patent Application Publication
(10) Pub. No.: US 2009/0326570 A1
(43) Pub. Date: Dec. 31, 2009

(54) Treated needle holding tube for use in tattooing

(76) Inventor: Kenneth Brown, Carson City, NV (US)

Correspondence Address: SILVERSKY GROUP LLC, 5422 LONGLEY LANE, SUITE B, RENO, NV, 89511, US

(21) Appl. No.: 12/215,588

(22) Filed: Jun. 27, 2008

(51) Int. Cl. A61B 17/34

(52) U.S. Cl. 606/186

(57) ABSTRACT

The present invention is directed to a device for use in tattooing, and more particularly to one or more hard coated or treated needle holding tubes to be used in tattooing human and/or animal skin. An ordinary tattooing needle holder, also referred to as a tattooing tube, of practically any shape or design, may be improved by treating select surfaces of the tattooing tube that are exposed to tremendous frictional force from an oscillating tattooing needle. The tubes herein disclosed exhibit significantly improved life-span, reduced friction, increased lubricity, and reduced material degradation over traditional tube designs, resulting in cost savings to a commercial tattoo artist and increased safety for recipients of tattoos.

### BRIEF DESCRIPTION OF THE INVENTION

The present invention is directed to a device for use in tattooing, and more particularly to one or more hard coated or treated needle holding tubes to be used in tattooing human and/or animal skin. An ordinary tattooing needle holder, also referred to as a tattooing tube, of practically any shape or design, may be improved by treating select surfaces of the tattooing tube that are exposed to tremendous frictional force from an oscillating tattooing needle. The tubes herein disclosed exhibit significantly improved life-span, reduced friction, increased lubricity, and reduced material degradation over traditional tube designs, resulting in cost savings to a commercial tattoo artist and increased safety for recipients of tattoos.

### CROSS-REFERENCES TO RELATED APPLICATIONS

Not Applicable.

### STATEMENT AS TO THE RIGHTS TO INVENTIONS MADE UNDER FEDERALLY SPONSORED RESEARCH OR DEVELOPMENT

Not Applicable.

### REFERENCE TO A “SEQUENCE LISTING,” A TABLE, OR A COMPUTER PROGRAM LISTING APPENDIX SUBMITTED ON A COMPACT DISK

Not Applicable.

### BACKGROUND OF THE INVENTION

Tattooing is performed by means of a sharp, small diameter needle or cluster of needles. The needle(s) are dipped in or otherwise supplied with tattoo pigment and then vibrated into or repeatedly inserted into the skin where the tattoo is desired. The needle is carried in a tubular holder which includes means for vibrating the needle or repeatedly extending and retracting the needle. Such a tubular holder may be referred to as a tube, a tattooing tube, a guide tube, a needle holding tube, or as a needle holder.

The basic design for a tattooing tube is fairly simple. Professional tattoo artists have used a simply designed tube for years; this traditional design can be seen in Yacowitz, U.S. Pat. No. 4,771,660. A tattooing tube such as Yacowitz generally has a main body portion many times thicker, or wider, than the needle itself, and a much narrower operating point which fits relatively tightly around the needle. The main body portion of the tattooing tube may have an opening slit, or trough, running most of its length (ending before the narrower operating point portion) that allows a user to easily and accurately insert the needle into its proper place within the tube without damaging the needle's delicate point. The tube is usually made of stainless steel or aluminum, but may also be made of nickel-plated iron, brass, bronze or a hard plastic. The prior art teaches that of these materials, stainless steel is preferred because it is durable, it is easily cleaned, and it does not corrode easily.

Such tattooing tubes are relatively inexpensive. They do, however, have a very limited life-span. Generally in a daily use situation, such as with a professional tattoo artist working in a retail shop, typical or traditional tubes last approximately four to six month on average—even with ongoing maintenance and care. The lifespan is limited because a great amount of friction is generated between the ordinarily metal surface of the tube's operating point and the rapidly oscillating needle. The operating point of the tube degrades over the course of months of daily use, and in fact much of the tube's material wears away. Naturally, the needle's material also degrades, but since needles are replaced frequently, the wear on needles is not as obvious as the wear on tubes. In addition to being costly to replace worn down tubes, a customer safety issue is involved. The metal materials used to form the tubes and needles wear away during use, with that worn away metal material travelling down the needles or dissipating into the air surrounding the tattooing operation, and entering the skin of the customer being tattooed. This dissipating tube metal phenomenon has not been extensively studied or reported on in the context of commercial tattooing, but experienced tattoo artists working with light-colored pigments have noted that the pigments turn grey over time, obviously as a result of ground metal material entering the pigment. The infusion of metal fragments into tissue in and around a tattoo is undesirable.

A further drawback to traditional homogeneous-material tattooing tubes is that such tubes do not perform optimally from a mechanical standpoint. When two similar materials are rubbed together, they tend to generated greater friction than is typically generated by appropriately dissimilar materials. This friction results in a wearing of the materials, as noted above, as well as the generation of heat (which can effect both the tattoo artist holding the tube and the skin of the customer), and result in the rougher operation of the needle (which can affect the quality of the tattoo artist's work).

The shape and/or design of tattooing tubes has been explored and improved overtime. Many tube designs are available, ranging from tubes designed to carry single needles to tubes designed to carry multiple needles; and ranging from open designs wherein one or more needles sit in a trough-like operating point to closed designs wherein one or more needles are fully enclosed within the operating point. But little thought and/or experimentation has been attempted in the field regarding the material composition of tattooing tubes, or regarding possible hardening techniques or treatments being used to improve performance of a tattooing tube.

### BRIEF DESCRIPTION OF THE SEVERAL VIEWS OF THE DRAWING

FIG. 1 is an example of a tattooing machine with which the disclosed hard coated or treated tattooing tube may be utilized;

FIG. 2 is an example of a traditional tattooing tube;

FIG. 3 is a side view of the tattooing tube shown in FIG. 2;

FIG. 4 is a bottom-up view of the tattooing tube shown in FIGS. 2 and 3;

FIG. 5 is an isometric view of another tattooing tube, additionally showing select surfaces of the tattooing tube which may be treated to improve performance;

FIG. 6 is an isometric view of another tattooing tube, additionally showing select surfaces of the tattooing tube which may be treated to improve performance;

FIG. 7 is an isometric view of another tattooing tube, additionally showing select surfaces of the tattooing tube which may be treated to improve performance;

FIG. 8 is an isometric view of another tattooing tube, additionally showing select surfaces of the tattooing tube which may be treated to improve performance;

FIG. 9 is an isometric view of another tattooing tube, additionally showing select surfaces of the tattooing tube which may be treated to improve performance; and

FIG. 10 is an isometric view of another tattooing tube design, additionally showing select surfaces of the tattooing tube which may be treated to improve performance.

### DETAILED DESCRIPTION OF THE INVENTION

The present invention is directed to a device for use in tattooing, and more particularly to one or more hard coated needle holding tubes to be used in tattooing human and/or animal skin. An ordinary tattooing needle holder, also referred to as a tattooing tube, of practically any shape or design, may be improved by hard coating select surfaces of the tattooing tube that are exposed to tremendous frictional force from an oscillating tattooing needle. The tubes herein disclosed exhibit significantly improved life-span, reduced friction, increased lubricity, and reduced material degradation over traditional tube designs, resulting in cost savings to a commercial tattoo artist and increased safety for recipients of tattoos. The tube itself may alternatively be referred to as a tattooing tube, a guide tube, or as a needle holder.

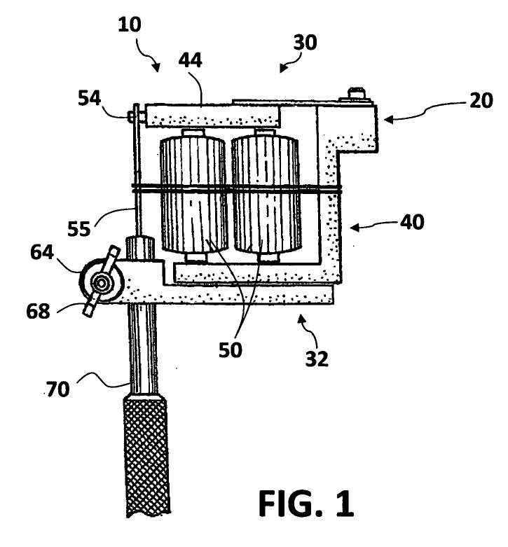

The herein disclosed hard coated tattooing tube(s) may be used with commercially available tattooing machine 10, one form of which is shown in FIG. 1. The tattooing machine 10 includes a U-shaped housing 20 placed on its side and having: an upper leg 30, a lower leg 32, and a connecting side leg 40. The upper leg 30 is made of a rigid armature bat 44 and a rearwardly projecting side leg 40. Electrical coils 50 are mounted on the housing 20 for use in causing the upper arm 30 to vibrate.

The leading end of the upper leg 30 and the front vertical surface of bar 44 carry a projecting pin 54 for coupling with a tattooing needle 55. The leading end of the lower leg 32 is formed with a horizontally disposed split ring 64 which is adapted to receive the needle holder of the invention and carries a threaded wing nut 68 for securing the tattooing tube 70 therein.

The tattooing machine 10 may and usually does include other parts which need not be described herein. Those skilled in the art will appreciate that other needle-driving, or tattooing, machines are available or may be devised. All that is required to be used with the present invention is an ability to securely hold a tattooing tube in place and an ability to drive, or oscillate, a tattooing needle within the tattooing tube.

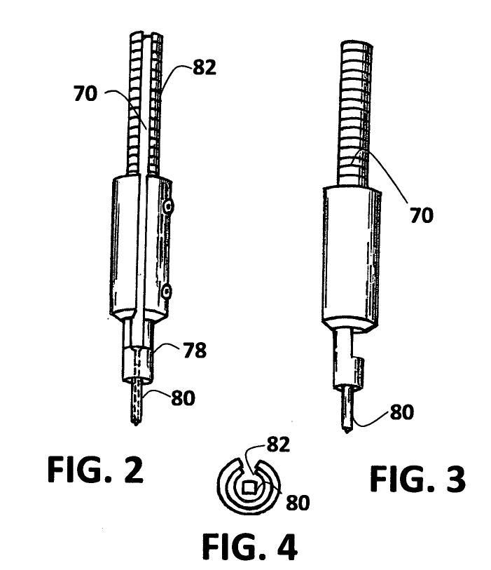

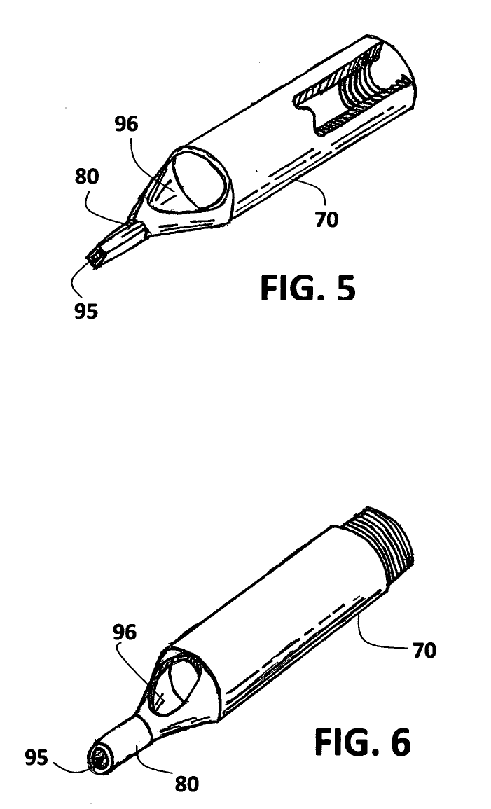

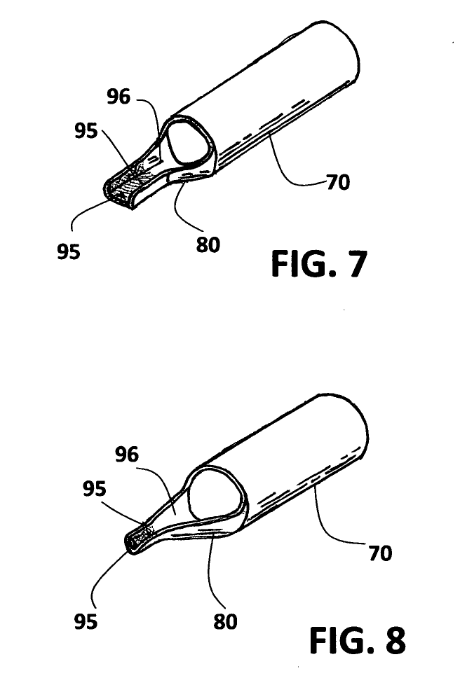

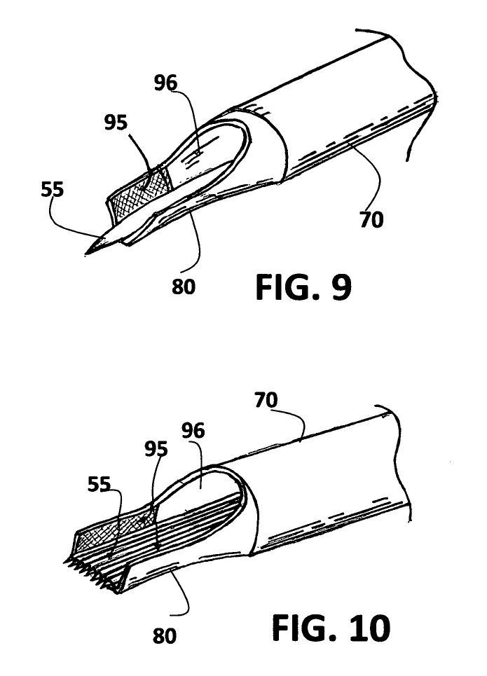

The herein disclosed hard coating, and hardening treatment, for tattooing tubes may be utilized with practically any tube shape design. As may been seen in FIGS. 2 through 4, a tattooing tube design known in the art is a hollow metal tube 70 of a suitable diameter having a slot 82, which may also be called a trough, in its wall extending from the upper end of the tube to near the lower end. FIG. 3 shows the same tattooing tube 70 as FIG. 2, but from a slightly different angle. FIG. 4 also shows the same tattooing tube 70 as in FIGS. 2 and 3, but from an angle looking up at the operating point of the tattooing tube 70. The portion of the lower end without the slot 82 includes a cylinder, which is not slotted, having a through-hole which may have a V-shaped upper opening 78. The tattooing tube's inner surface formed by the through-hole may be referred to as the through-hole's surrounding surface. The lower portion of the tattooing tube 70, which may be referred to as operating point 80, shown in FIGS. 2 through 4 is only one example of how such a tube may be designed. As may be seen in FIGS. 5-10, the operating point 80 of a tattooing tube 70 may be designed in any number of ways. For example, operating point 80 may be squared-shaped and enclosed, as in FIG. 5. Operating point 80 may be round and enclosed, as in FIG. 6. Operating point 80 may instead be V-shaped and open, as in FIGS. 8 and 9. Operating point 80 may instead be wider, flat, and open as in FIG. 7 and FIG. 10. The figures provided and described herein are not meant to be an exhaustive list of the possible shapes and/or designs for tattooing tubes; they are only provided as examples. The purpose of the disclosure is not to provide improved tattooing tube shapes, layouts, or designs. The purpose instead is to provide for improved material composition and material characteristics of known tattooing tube designs by various methods of hardening treatments. As such, any shape or design of tattooing tube, whether previously known in the art or to be designed in the future, may be improved by utilizing the herein disclosed invention.

It is the interior surfaces of the operating point of the tattooing tube to which the herein disclosed hardening treatment is applied. Specific portions of tattooing tubes may be treated in order to provide superior life-span, decrease friction in its interaction with a tattooing needle, increase corrosion resistance, increase wear resistance, and increase lubricity. Many different types of hardening treatments are possible, and the treating may be implemented with one of several appropriate methods. The methods to be used include electroplating, physical vapor deposition (“PVD”), and chemical vapor deposition (“CVD”), each of which is a method known in the art for treating or hard coating a surface. Each of these methods may be utilized to deposit a layer of metal or other material having a desired property (increased durability, decreased friction, etc.) onto a surface lacking that property.

Electroplating, for example, is a process of using electrical current to reduce metal cations in a solution and coat a conductive object, in this case the metallic tattooing tube, with a relatively thin layer of the metal. Physical vapor deposition, for example, may be used to coat a surface or a portion of a surface with non-metalic materials and involves depositing thin films of the coating material onto a surface by vaporizing the coating material and then condensing that vaporized material onto the desired surface. Chemical vapor deposition may also be used and, for example, involves applying one or more volatile precursors to the desired surface. The precursors react and/or decompose on the surface, producing the desired hardening and other characteristics.

One or more surfaces or portions of a tattooing tube may be hard coated with hard chrome performed by electro-plating, with diamond suspended nickel performed by electro-plating, with poly-crystalline diamond performed by chemical vapor deposition or physical vapor deposition, with titanium nitride (also called “tin”) performed by physical vapor deposition, with cubic boron nitride (also called “CBN”) performed by physical vapor deposition, with nano composition performed by physical vapor deposition, with nano ceramic performed by physical vapor deposition, with titanium carbo nitride (also called “TiCN”) performed by physical vapor deposition, or with titanium aluminum nitride (also called “TIAN”), performed by physical vapor deposition. Each of these hard coating methods may be used to create tattooing tubes with the desired improved characteristics. Each of these coatings combined with the coating method will be understood by those skilled in the art.

Alternatively, materials with the desired characteristics may be physically bonded to the desired surfaces of the tattooing tubes in order to improve durability and reduce friction. Any type of stone may be bonded to the frictional surfaces. Additionally, any type of ceramic may be bonded to the frictional surfaces. More generally, any type of crystalline-structured material may be bonded to the frictional surfaces to improve durability, reduce friction, and/or increase lubricity. Many types of bonding methods may be utilized. For example, electric ceramic bonding involves applying a high voltage and heat between the ceramic coating and the frictional surfaces of the tattooing tube. Those skilled in the art will appreciate that other methods may be used to physically bond stone or ceramic material to the frictional surfaces of tattooing tubes.

As another alternative method of creating harder, more durable, and lower friction tattooing tube frictional surfaces, various heat treatment methods may be applied. For example, selected surfaces of a tattooing tube may be cooled more quickly than remaining surfaces in order to increase the hardness of the selected surfaces. This may be accomplished by insulating the remaining surfaces, with clay for example, while the selected frictional surfaces of the tattooing tubes are allowed to quickly cool after the forging process. The quickly cooled surfaces will exhibit increased hardness. Those skilled in the art will appreciate that other methods may be used to heat treat tattooing tubes or selected surfaces of tattooing tubes.

Yet another appropriate treatment method to increase durability and decrease friction is to impregnate the frictional surface with a lubricant. Impregnating specific surfaces on a needle holder with a biocompatible lubricant will increase those surfaces' lubricity in their interaction with the rapidly oscillating tattooing needle, and thus will increase durability and decrease friction. Those skilled in the art will appreciate that other lubricants and lubrication methods may be appropriate.

For the purposes of this application, the term “treating” (or “treated”) should be read to encompass all the hardening techniques discusses above, including bonding stone, ceramic, or other materials to a metallic tattooing tube and including various heat treatment methods applied to a metallic tattooing tube, as well as any other methods that might exist or be developed in the future that serve a similar function. Of course, the term “treating” also includes each of the hard coating techniques described herein (electroplating, physical vapor deposition, chemical vapor deposition, etc.) as well as depositing of one or more layers of dissimilar material onto select surfaces of a needle holder or impregnating the select surfaces with a lubricant.

Looking to the figures, specific portions of the various representative tattooing tube designs will be pointed out that optimally should be treated. In FIG. 2, the inside surface of operating point 80, and the surfaces of through-hole 78, including the V-shaped portion, may be treated. It is these surfaces that constantly interact with a tattooing needle during a tattooing operation, and so these surfaces should be treated in order to increase the tattooing tube's durability (and thus increased life-span), increase the tattooing tube's lubricity to reduce the friction force resulting from the repetitive movement of the tattooing needle, and reduce the tattooing tube's degradation by increasing the corrosion resistance of the selected surfaces. In FIGS. 5 and 6, it is inside surface 95 of operating point 80 that may be treated to improve performance. Additionally, nozzle inside surfaces 96 in both FIG. 5 and 6, the inside surfaces of the narrowing portion of the tattooing tube just above operating point 80, may be treated because nozzle 96 also must withstand frictional force applied by the oscillating tattooing needle, although to a lesser extent than operating point 80. In FIGS. 7 and 8, the inside surfaces at 95 (cross-hatched in the figures so as to be visually apparent), of operating point 80 may be treated. FIG. 7 represents a wide, flat bottom operating point tattooing tube design, and treated surfaces 95 include the two side walls of operating point 80 as well as the bottom flat surface. Nozzle inside surfaces 96 may also be treated, although as explained, treating of nozzle 96 is less important as it is faced with relatively less frictional force than the inside surfaces 95 of operating point 80. FIG. 8 is very similar to FIG. 7 except that FIG. 8 is a V-shaped operating point 80; surfaces 95 of operating point 80 may be treated, and nozzle inside surfaces 96 may additionally be treated. FIG. 9 is similar to FIG. 8 with a tattooing needle 55 shown lying within the tattooing tube. Inside surfaces 95 of operating point 80 may be treated, and nozzle inside surfaces 96 may additionally be treated. Finally, FIG. 10 is similar to FIG. 7 with multiple tattooing needles 55 shown lying within the tattooing tube. Inside surfaces 95 of operating point 80, including both side walls and the flat bottom portion, may be treated, and nozzle inside surfaces 96 may additionally be treated.

The above discussed hard coating, bonding, and treating methods may be applied to select surfaces of a tattooing tube. Selecting specific surfaces, as opposed to treating an entire tattooing tube, is preferable because doing so is less expensive than treating an entire tattooing tube and results in an overall lighter-weight tattooing tube. Nevertheless, the discussed techniques may be applied to every surface of a tattooing tube if so desired. Such a tattooing tube, fully treated, would be functionally equivalent, although more expensive and heavier, and is intended to be within the scope of this specification. Furthermore, any combination of treated and non-treated surfaces is also intended to be within the scope of this specification.

While the present invention has been illustrated and described herein in terms of a preferred embodiment and several alternatives associated with hard coated or treated tattooing tubes for use in professional and/or commercial tattooing operations, it is to be understood that the various components of the combination and the combination itself can have a multitude of additional uses and applications. For example, the hard coated needle holder for tattooing herein disclosed can easily be adapted to other settings or uses such as tattooing animal skins or hides, scientific research, or livestock husbandry. Accordingly, the invention should not be limited to just the particular descriptions and various drawing figures contained in this specification that merely illustrate one or more preferred embodiments and applications of the principles of the invention.

What is claimed is:

1. An apparatus for holding a tattooing needle during a tattooing operation, comprising a needle holder having one or more treated surfaces that contact the tattooing needle.

2. The apparatus as recited in claim 1, wherein the needle holder includes an operating point having an interior surface that contacts the tattooing needle, and wherein the one or more treated surfaces include the interior surface.

3. The apparatus as recited in claim 2, wherein the needle holder further includes a nozzle inside surface that contacts the tattooing needle, and wherein the treated surfaces additionally include the nozzle inside surface.

4. The apparatus as recited in claim 1, wherein the needle holder includes an operating point having an inside surface forming a through-hole having a surrounding surface, and wherein the treated surfaces include the inside surface of the operating point and the surrounding surface of the through-hole.

5. The apparatus as recited in claim 4, wherein the needle holder further includes a nozzle inside surface that contacts the tattooing needle, and wherein the treated surfaces additionally include the nozzle inside surface.

6. The apparatus as recited in claim 1, wherein the treated surfaces are formed by electroplating, physical vapor deposition, or chemical vapor deposition.

7. The apparatus claim as recited in claim 1, wherein the treated surfaces are formed of a crystalline structure.

8. The apparatus claim as recited in claim 1, wherein the treated surfaces are formed of a layer of a crystalline structured material.

9. The apparatus claim as recited in claim 1, wherein the treated surfaces are formed by impregnation of a lubricant.

10. The apparatus as recited in claim 1, wherein the treated surfaces are formed by physically bonded material.

11. The apparatus as recited in claim 1, wherein the treated surfaces are formed by heat treatment.

12. The apparatus as recited in claim 11, wherein the heat treatment involves cooling an area of material of the needle holder more quickly than other material forming the needle holder.

13. The apparatus as recited in claim 1, wherein the treated surfaces are formed of a layer of material having an increased durability and a smoother surface than other material forming the needle holder.

14. The apparatus as recited in claim 1, wherein the treated surfaces are formed of a layer of a material having an increased durability than other material forming the needle holder.

15. The apparatus as recited in claim 1, wherein the treated surfaces are formed of a layer of a material having a smoother surface than other material forming the needle holder.

16. The apparatus as recited in claim 1, wherein the treated surfaces are formed of a layer of a material having increased lubricity in its interaction with the tattooing needle relative to other material forming the needle holder.

17. The apparatus as recited in claim 1, wherein the treated surfaces are formed of a layer of hard chrome, diamond suspended nickel, poly-crystalline diamond, titanium nitride, cubic boron nitride, nano composition, nano ceramic, titanium carbo nitride, titanium aluminum nitride, ceramic or stone.

18. The apparatus as recited in claim 17, wherein the layer of stone is physically bonded onto the needle holder.

19. The apparatus as recited in claim 17, wherein the layer of ceramic is physically bonded onto the needle holder.

20. The apparatus as recited in claim 19, wherein the layer of ceramic is physical bonded through an application of a high voltage and an amount of heat between the needle holder and the layer of ceramic.

21. An apparatus for holding a tattooing needle during a tattooing operation, comprising a needle holder having one or more treated surfaces that contact the tattooing needle, the needle holder including an operating point, the operating point having an interior surface that contacts the tattooing needle and has a flat bottom and two side walls.

22. The apparatus as recited in claim 21, wherein the treated surfaces include the flat bottom and the two side walls.

23. The apparatus as recited in claim 22, wherein the needle holder further includes a nozzle inside surface that contacts the tattooing needle, and wherein the treated surfaces include the nozzle inside surface.

24. An apparatus for holding a tattooing needle during a tattooing operation, comprising a needle holder having one or more treated surfaces that contact the tattooing needle, the needle holder including an operating point, the operating point having a V-shaped interior surface that contacts the tattooing needle.

25. The apparatus as recited in claim 24, wherein the treated surfaces include the V-shaped interior surface.

26. The apparatus as recited in claim 25, wherein the needle holder additionally includes a nozzle inside surface, and wherein the treated surfaces include the nozzle inside surface.

27. A method for altering a needle holder to make one or more surfaces of the needle holder more durable, comprising the steps of:
selecting the surfaces of the needle holder; and
treating the surfaces.

28. The method as recited in claim 27, wherein the step of treating includes the step of electroplating the surfaces, applying a physical vapor deposition to the surfaces, or applying a chemical vapor deposition to the surfaces.

29. The method claim as recited in claim 27, wherein the step of treating includes the step of impregnating the surfaces with a lubricant.

30. The method claim as recited in claim 27, wherein the step of treating includes the step of depositing a layer of crystalline structured material onto the surfaces.

31. The method as recited in claim 27, wherein the step of treating includes the step of physically bonding a material to the surfaces.

32. The method as recited in claim 27, wherein the step of treating includes the step of heat treating the surfaces.

33. The method as recited in claim 32 wherein the step of heat treating includes the step of cooling an area of material of the needle holder more quickly than other material forming the needle holder.

34. The method as recited in claim 27, wherein the step of treating includes the step of depositing a layer of material onto the surfaces, the material being dissimilar from a material forming the needle holder.

35. The method as recited in claim 27, wherein the step of treating includes the step of depositing a layer of material onto the surfaces that has a greater durability than other material forming the needle holder.

36. The method as recited in claim 27, wherein the step of treating includes the step of depositing a layer of a material that has a smoother surface than other material forming the needle holder.

37. The method as recited in claim 27, wherein the step of treating includes the step of depositing a layer of a material that has increased lubricity in its interaction with a tattooing needle relative to other material forming the needle holder.

38. The method as recited in claim 27, wherein the step of treating includes the step of depositing a layer of hard chrome, diamond suspended nickel, poly-crystalline diamond, titanium nitride, cubic boron nitride, nano composition, nano ceramic, titanium carbo nitride, titanium aluminum nitride, stone or ceramic onto the surfaces.

39. The method as recited in claim 38, wherein the step of depositing includes the step of physically bonding the layer of stone to the surfaces.

40. The method as recited in claim 38, wherein the step of depositing includes the step of physically bonding the layer of ceramic to the surfaces.

41. The method as recited in claim 40, wherein the step of physical bonding includes the step of applying a high voltage and an amount of heat between the needle holder and the layer of ceramic.
